(19) United States
(12) Patent Application Publication
(10) Pub. No.: US 2026/0363881 A1
(43) Pub. Date: Nov. 19, 2026

(54) Hybrid Gaze / EMG Interface for Upper Limb Prosthesis

(71) Applicant: THE JOHNS HOPKINS UNIVERSITY, Baltimore, MD (US)

(72) Inventor: Rebecca Jamie Greene, Baltimore, MD (US)

(21) Appl. No.: 19/680,594

(22) Filed: May 18, 2026

Related U.S. Application Data
(60) Provisional application No. 63/807,235, filed on May 16, 2025.

(51) Int. Cl. A61F 2/72; A61F 2/54; A61F 2/70; A61F 4/00; G06F 3/01

(52) U.S. Cl. CPC A61F 2/72; A61F 2/54; A61F 4/00; A61F 2002/704; G06F 3/013; G06F 3/015

(57) ABSTRACT

Shared control has emerged as a promising alternative system paradigm for prosthetic device movement. In the present invention, control is distributed between the user and an algorithm that predicts the user's motion intent. Thus, the controller can manage low-level motion planning and allow the user to concentrate on high-level decisions, such as reaching and grasping. The majority of this work has examined use of computer vision (CV) systems to classify objects and determine optimal grasp type. The control algorithm can be paired with gaze tracking, pose tracking, and EMG sensors to gather data to improve the predictive nature of the present invention. In some embodiments, the algorithm can leverage dynamic movement primitives to generate smooth trajectories between a rest position and a desired movement endpoint.

### CROSS REFERENCE TO RELATED APPLICATIONS

This application claims the benefit of U.S. Provisional Patent Application No. 63/807,235 filed on May 16, 2025, which is incorporated by reference, herein, in its entirety.

### FIELD OF THE INVENTION

The present invention relates generally to prosthetics. More particularly, the present invention relates to a hybrid gaze-EMG interface for a limb prosthesis.

### BACKGROUND OF THE INVENTION

Prosthetic non-wear has been reported in 20% of the adult upper-limb loss population and rejection rates range from 35-40% for myoelectric and cable-controlled systems, with ‘lack of function’ being cited as the main cause of disuse. 88% of upper limb non-prosthetic users claim that the devices are “mentally and physically tiring” to use, while 98% of non-prosthetic users reported being “just as, if not more functional without a prosthesis”. It is important to note that the issues with prosthesis functionality are not due to lack of capability with the mechatronics. Studies in robotic grasping have shown that purely mechanical systems are capable of dexterous object manipulation. The Applied Physics Lab at Johns Hopkins University built a 26DOF upper limb prosthetic to fully emulate the joint motion of a biological limb, but subjects using this device have only utilized at most 3DOF. Commercially available prosthetic devices have at most 2-powered-DOF, typically wrist rotation with discrete hand grasps, occasionally with flexion as a 3rd passive joint. There is a substantial difference in dimensionality between the usable configuration space for a prosthesis user's hybrid human-robot serial manipulator and that of a purely biological or robotic arm. This dexterity gap indicates that issues with prosthesis proficiency stem from the human-robot interface and the ability to reliably interpret and execute user intent.

The current paradigm for clinically available powered prosthesis is a feed-forward controller based on classification of motion intent. Electromyogram (EMG) signals are generated by contraction of remaining musculature in the residual limb and can be measured noninvasively by placing electrodes on the surface of the skin. Today, machine learning-based pattern-recognition (PR) algorithms are considered the state of the art in research-. These systems classify EMG signals into a predefined set of motion classes. Pattern-recognition classifiers have seen increasing use in clinical settings, although they are still limited in the number of available DOF—typically 1DOF in the wrist (pronation/supination) is combined with a hand that is capable of performing multiple discrete grasps (e.g. ‘hand open’, ‘power’, ‘tripod’, etc.). Further, the PR strategy necessitates sequential control of each individual joint. Simultaneous control strategies are being investigated, however none have proven sufficiently reliable for clinical use.

Managing even a single prosthetic joint with an EMG-only interface strategy imposes a substantial cognitive burden on the user compared to a biological limb. The extent of this cognitive load increases with the number of extrinsic DOF to be controlled. Even when presented with multiple controllable DOF, subjects using interfaces that require sequential joint modulation tend to utilize just one joint, leading to inefficient and unnatural motion. Ultimately this unnatural compensatory motion leads to long-term overuse injuries, which are a significant health concern that affects many amputees and can lead to a significant decrease in quality of life. Reasons for compensatory motion include lack of joint function in prosthetic devices (especially missing DOF in the wrist), lack of confidence or reliability in the controllable joints that do exist, as well as physiological strategies to manage the extra weight of a prosthetic device as compared to a biological limb. These factors, in combination with heavy mental load are cited as the top reasons contributing to prosthesis abandonment.

One of the key promises of semi-autonomous systems, especially for the upper limb prosthesis application, is that the controller manages low level joint angle planning while the user retains task-level decision making. Because the biological and mechanical joints are linked serially, traditional approaches to cooperative tasks (like impedance-based controllers) are not applicable. Thus, the design of appropriate interfaces is a significant and compelling challenge whose outcome has a high impact on the quality of life for patients.

Conventional approaches to powered prosthesis control rely on decoding electromyographic (EMG) signals in the residual limb that are generated as a result of muscle contractions. Machine learning classifiers enable sequential DOF modulation, or alternatively regression algorithms support simultaneous control of up to 7 DOF. However, these solutions are limited by the non-stationary nature of EMG signals due to factors like loading conditions or user fatigue, which are compounded by the challenge of maintaining consistent electrode placements in a sweaty silicone socket. Even with a perfect classifier, many users still experience high cognitive demands when managing several DOFs with an EMG-only interface strategy.

It would therefore be advantageous to provide a hybrid gaze-EMG interface for a limb prosthesis to ease the mental burden on the prothesis user and facilitate prosthesis adoption.

### SUMMARY OF THE INVENTION

The foregoing needs are met, to a great extent, by the present invention, wherein one aspect is a system comprising an electromyography (EMG) sensor, a gaze tracker, and a pose tracker. The system also includes a processing device, wherein the processing device is programmed to receive data from the EMG sensor, the gaze tracker, and the pose tracker, such that the processing device generates a trajectory for a prosthetic device from the data from the EMG sensor, the gaze tracker, and the pose tracker.

In accordance with an aspect of the present invention, dynamic motion primitives (DMPs) are used to generate the trajectory for the prosthetic device. The prosthetic device can take the form of a transhumeral prosthesis. The prosthetic device can take the form of an upper limb prosthesis. The trajectory can be determined in a task-based space.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates perspective view of a hybrid gaze-EMG prosthesis interface being used to complete the experimental task.

FIG. 2 illustrates a schematic view of a hybrid gaze-EMG prosthesis interface being used to complete the experimental task along with bio-aware path planning and visual grasp prehension.

FIG. 3 illustrates a schematic diagram of hardware and data acquisition, intent interpretation, and motion command, according to an embodiment of the present invention.

FIG. 4 illustrates perspective views of components of a system according to an embodiment of the present invention.

FIG. 5 illustrates a schematic diagram of a transradial virtual modular prosthetic limb (vMPL) that can be modeled as a 3DOF wrist with a 1DOF hand.

FIGS. 6A and 6B illustrate system diagrams for an SA controller, according to an embodiment of the present invention.

FIG. 7 illustrates a flow diagram of a workflow of the user interface with the SA controller.

FIG. 8 illustrates a schematic diagram of the pick-and-place task.

FIG. 9 illustrates a graphical view of task completion rates.

FIG. 10 illustrates graphical views of ranges of motion according to completion of tasks according to an embodiment of the present invention.

FIG. 11 illustrates graphical views of a ratio of pose changes in the wrist.

FIG. 12 illustrates graphical views of sample paths through joint angle space for different task types with both the 3DOF SA and PR controller.

FIG. 13 illustrates a graphical view of joint utilization, according to an embodiment of the present invention.

FIG. 14 illustrates a schematic view of a setup for the present invention.

FIG. 15 illustrates a schematic diagram of the controller and offline learning process.

FIG. 16 illustrates graphical views of linearity of RBF weights.

FIG. 17 illustrates a graphical view of an exemplary distribution of unnormalized RBFs (i.e. φᵢ(s)s(cᵢ)).

FIG. 18 illustrates graphical views of Overall Generalization Performance.

FIG. 19 illustrates graphical views of a trajectory reproduction example.

FIG. 20 illustrates graphical views of sample trajectories from the online experiment.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

Shared control has emerged as a promising alternative system paradigm for prosthetic device movement. In the present invention, control is distributed between the user and an algorithm that predicts the user's motion intent. Thus, the controller can manage low-level motion planning and allow the user to concentrate on high-level decisions, such as reaching and grasping. The majority of this work has examined use of computer vision (CV) systems to classify objects and determine optimal grasp type. The control algorithm can be paired with gaze tracking, pose tracking, and EMG sensors to gather data to improve the predictive nature of the present invention. In some embodiments, the algorithm can leverage dynamic movement primitives to generate smooth trajectories between a rest position and a desired movement endpoint.

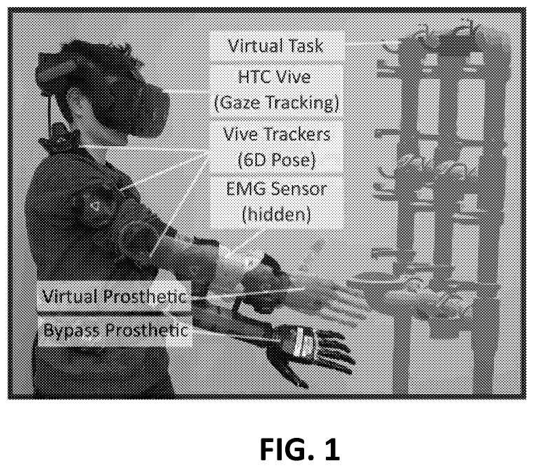

Semi-autonomous prostheses allow for robust object interaction. Initial iterations in this body of work revolved around the use of camera-based image recognition systems to identify object types and look up appropriate grasp type in response to a ‘close’ EMG command. Other more empirical approaches have utilized sensorized hands to try to maximize the contact area between the hand and objects for more optimal grasping. In the past 5 years, additional projects have started to include powered wrist rotation as a controllable DOF and would additionally rotate the wrist to maintain user-set wrist angles or the output of a pose recognition system. Semi-autonomous systems exploring wrists with 2DOF have determined wrist angles based on predetermined poses for each recognized object or again to maintain user-set angles. All of these systems use proximity to determine target objects for the semi-autonomous algorithms. This can be an effective method for transradial amputees, who retain enough DOF in the shoulder and elbow to determine palm position, but does not extend well to patients with higher levels of amputation, who arguably have more to gain from semi-autonomous systems. Previously, a novel multi-modal interface for a semiautonomous prosthesis controller utilized an estimated gaze vector to determine target objects in a virtual reality (VR) experimental setup, determining grasp type and 1DOF wrist orientation based on object type and pose. In the present invention, a refined version of the interface allows subjects to perform a series of pick and place tasks using different prosthetic systems, as illustrated in FIG. 1.

FIG. 1 illustrates perspective view of a hybrid gaze-EMG prosthesis interface being used to complete the experimental task. Participants pick up a cylinder and transport it to a corresponding holder. The gaze vector can be used to select a target object or holder, and EMG signals can be used to move the prosthetic wrist along a calculated path to align with the target. During the experiment, subjects wore an EMG sensor under a bypass socket for a commercial prosthesis. Although the motion of the physical prosthesis did not directly effect task completion, it was important to consider changes in user behavior due to the inertia. Kinematic trackers were used to measure the motions of the biological limb. Most of the experiment took place in a VR environment rendered by the VIVE headset.

As a part of the present invention, Semi-Autonomous (SA) and Pattern Recognition (PR) systems with varying wrist DOF were used to perform functional tasks in a virtual environment. Changes in the available DOF were observed to determine the impact on performance metrics related to device functionality and workspace accessibility. It was observed that the present invention improves the state of the art in at least four ways. 1) This is the first semi-autonomous upper limb prosthetic study to consider a 3DOF wrist that fully emulates the functionality of a biological wrist. 2) This is the first semi-autonomous upper limb prosthesis study to examine how variations in the number of controllable DOF impact the user. 3) The results shown herein highlight the importance of semi-autonomous systems in high-DOF man-machine systems. 4) The results show that with semi-autonomous systems, there is now motivation to increase the available powered DOF when building wrists for upper limb prosthesis.

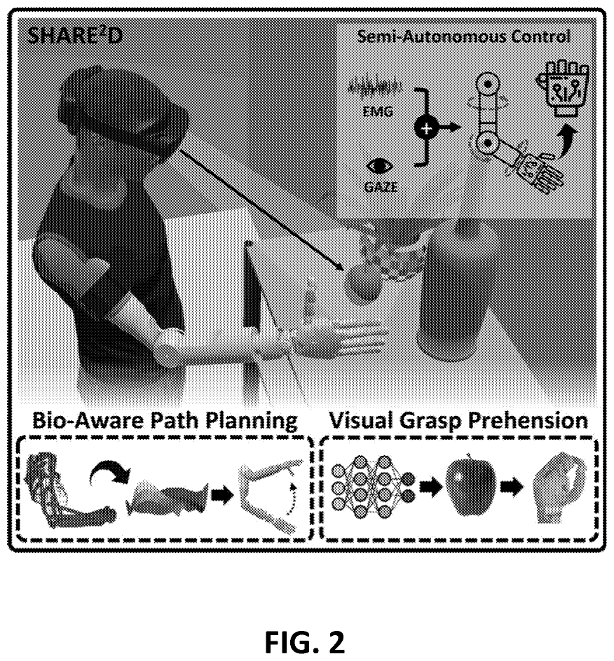

FIG. 2 illustrates a schematic view of a hybrid gaze-EMG prosthesis interface being used to complete the experimental task along with bio-aware path planning and visual grasp prehension. As illustrated in FIG. 2, the present invention enables the user to look at the target object, here an orange, and retrieve that target object with system support. EMG and gaze data are used to control the path of the prosthetic limb. The present invention thereby leverages bio-aware path planning and visual grasp prehension to enable the movements of the prosthetic.

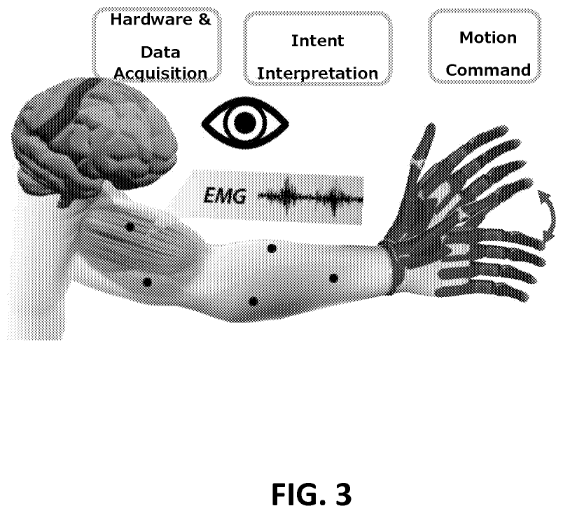

FIG. 3 illustrates a schematic diagram of hardware and data acquisition, intent interpretation, and motion command, according to an embodiment of the present invention. Here, the hardware of the system allows for data acquisition. The data is at least in the form of gaze tracking data, environment data, and EMG data, though additional data known to or conceivable to one of the art can also be used. The data is used to interpret intent in the environment into motion command of the prosthesis.

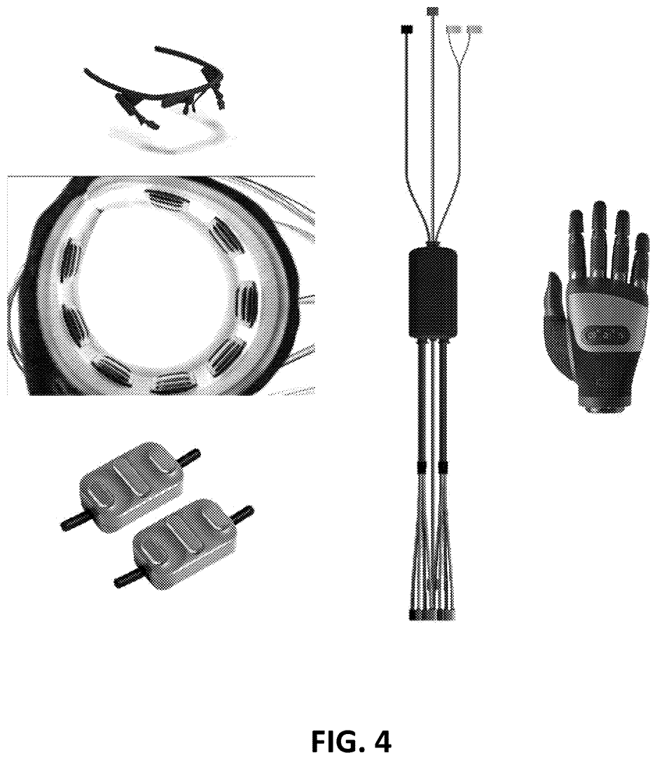

FIG. 4 illustrates perspective views of components of a system according to an embodiment of the present invention. As illustrated in FIG. 4, the system includes a head mounted gaze tracking device, here in the form of glasses, sensors for gathering and receiving EMG data, and a prosthetic configured to receive movement-generating input.

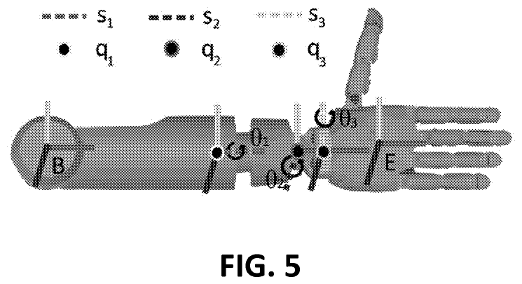

FIG. 5 illustrates a schematic diagram of a transradial virtual modular prosthetic limb (vMPL) that can be modeled as a 3DOF wrist with a 1DOF hand. B and E denote the base and palm coordinate frames, respectively. The points qᵢ and vectors sᵢ define axes of rotation for each joint. θ₁ rotates the palm around the axis of the forearm, θ₂ sets wrist deviation, and θ₃ corresponds to flexion/extension. A single variable θₕ (not pictured) sets the angle for all 19 finger joints to determine the aperture of the hand.

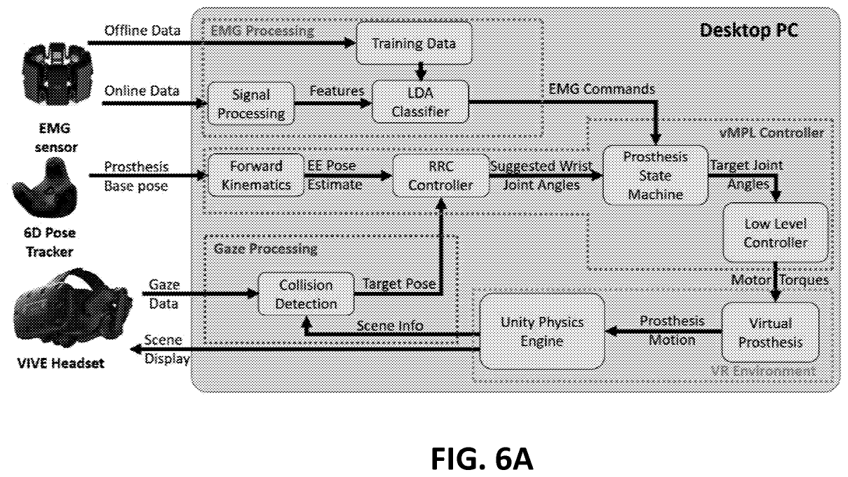

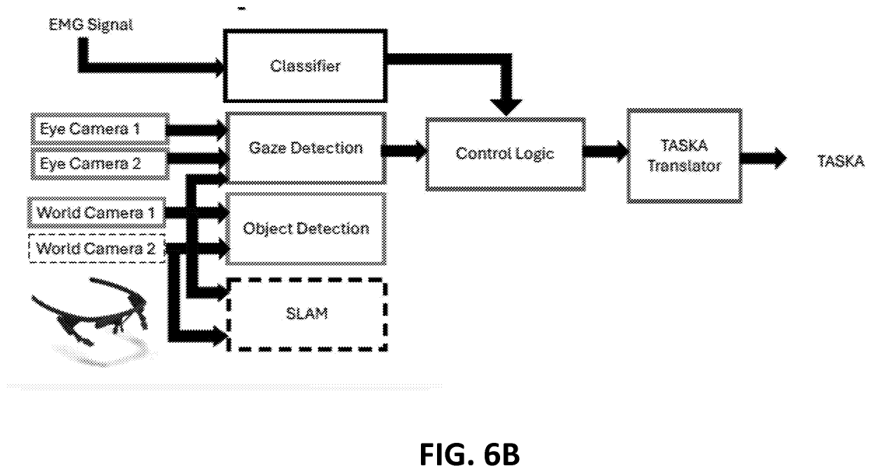

FIGS. 6A and 6B illustrate system diagrams for an SA controller, according to an embodiment of the present invention. Collisions between gaze vectors sensed from the VIVE headset and features in the VR environment are used to determine the user's desired pose for the prosthetic hand. The location of the base of the prosthetic is determined with a kinematic tracker, and the actual joint angles of the device are used to estimate the current hand pose. Depending on the interface type (PR or SA) and user EMG commands, the target prosthesis joint angles may be those suggested by an algorithm of the present invention or come directly from the user.

The vMPL is modelled after the Modular Prosthetic Limb (MPL) from the JHU APL. The MPL is a 26DOF manipulator that includes 3DOF at the shoulder, 1DOF at the elbow, 3DOF at the wrist, and 19DOF in the hand. For use as a transradial prosthetic, only the lower 22DOF are used, defining a base at the bottom of the elbow joint, as illustrated in FIG. 5. An example of the present invention using more of the functionality of the MPL will also be described further herein, with respect to an upper limb prosthesis. These 22DOF are divided into three wrist joints θ_{1:3} that define gross motion of the palm, and 19 hand joints θₕ that define the more dexterous configuration of the fingers.

The vMPL and the rest of the Virtual Reality (VR) environment is constructed in Unity 2018 (Unity Software Inc, San Francisco CA, USA) as a series of rigid bodies connected by motor driven hinge joints. Masses for the rigid bodies are set proportionally to the masses of the corresponding parts in the MPL, A low level proportional controller (the ‘vMPL arbiter’) is used with the vMPL to allow for an abstraction of positional control of θ_{1:3} and classification control of θₕ. Inputs to the vMPL arbiter consist of target joint angles for the upper arm joints, and a hand class for the lower arm joints. Given a set of target angles, the vMPL controller uses a velocity-limited proportional controller to drive each of the modeled motors:

$${θ}_{t} = {θ}_{t - 1} + \frac{{K}_{θ} {Δ}_{t}}{{e}_{max}} ( max ( {e}_{max} , min ( - {e}_{max} , {θ}_{t}^{*} - {θ}_{t - 1} ) ) ) \qquad ( 1 )$$

where t is the timestep, K_{θ} is the maximum joint angle velocity, Δₜ is the control loop period, eₘₐₓ is a scaling factor, and θ* is the target angle for the joint.

After using this layer of abstraction, now the vMPL can be modeled as a 4DOF system with a mobile base, as illustrated in FIG. 5. Using screw theory, the pose of the prosthetic palm is expressed in the global coordinate frame (gₛₜ) as a function of the wrist angles and the pose of the base:

gₛₜ(θₐ,t)=g_{sb}(t)e^{ŝ}^{₁}^{θ}^{₁}e^{ŝ}^{₂}^{θ}^{₂}e^{ŝ}^{₃}^{θ}^{₃}g_{bt} (2)

g_{sb}(t) is the transform between the coordinate frame at the base of the prosthesis and the global coordinate frame of the VR system, calculated using kinematic trackers. Ŝ₁, Ŝ₂, Ŝ₃ are skew-symmetric matrix representations of the screw axis S₁, S₂, S₃. Each screw axis is a normalized twist, with values v and w corresponding to the linear and angular velocity components respectively.

$${S}_{I} [ \begin{matrix}{w}_{i} \\ {v}_{i}\end{matrix} ] = [ \begin{matrix}{s}_{i} \\ {q}_{i} \times {s}_{i} + {h}_{i} * {s}_{i}\end{matrix} ] \qquad ( 3 )$$

Here hᵢ is the ratio of linear to angular velocity for the twist. Since all of the prosthetic joints are purely rotational, hᵢ=0∀i∈[1,3]. For each joint i, the point qᵢ and vector sᵢ define the axis of rotation as shown in FIG. 5.

Although the experimental task was completed with a virtual prosthetic, subjects were outfitted with a commercial prosthetic system to account for changes in behavior due to the device weight. This setup included a bebionic3 prosthetic hand (Ottobock, Duderstadt, Germany) and Motion Control Wrist Rotator (Fillauer LLC, Chattanooga, TN), mounted on a bypass socket that allowed the non-amputee subjects to mount the prosthesis on their forearm. EMG data was acquired using a Myo Armband EMG sensor (Thalmic Labs, Ontario, CA).

To render the VR environment, the system uses the VIVE Pro Eye head-mounted display (HMD) (HTC Corporation, New Taipei City, Taiwan), as illustrated in FIG. 1. The HMD also contains internal eye-tracking hardware that allows the system to measure visuomotor behavior such as gaze direction and visual attention (Tobii Technology AB, Stockholm, Sweden). HTC VIVE trackers (HTC Corporation, New Taipei City, Taiwan) were mounted on both hips as well as the shoulder, upper arm, elbow, forearm, and wrist of a participant's dominant limb to record the trunk and arm kinematics. 6D pose information was sampled from each tracker at 90 Hz. When calculating outcome metrics 3D position data was filtered in both the forward and backwards directions using a 6-Hz second-order Butterworth low-pass filter. Orientation data was filtered using spherical linear interpolation of the pose quaternions. The forearm tracker was also used to superimpose the virtual prosthetic on top of the user's limb for the experimental task.

The eight bipolar electrodes on the EMG sensor were sample at 200 Hz. No digital signal filtering was necessary due to front-end EMG signal conditioning incorporated into the armband. For each EMG channel, five time-domain signals (mean absolute value, variance, waveform length, zero crossings, and slope sign change) were extracted from 250 ms windows, with a 50 ms step between windows, as is standard in the literature. This led to a total of 40 features per time window. At the beginning of each experimental session, subjects trained a Linear Discriminant Analysis (LDA) classifier to translate EMG signals into commands to the prosthesis.

The LDA algorithm calculates a new set of basis vectors for the labeled dataset, chosen to minimize within-class scatter

$${σ}_{W}^{2}$$

while maximizing inter-class scatter

$${σ}_{B}^{2}$$

within the training data.

$${σ}_{W}^{2} = {\sum }_{c = 1}^{C} {\sum }_{i = 1}^{{N}_{c}} ( {x}_{c i} - {μ}_{c} ) {( {x}_{c i} - {μ}_{c} )}^{T} \qquad ( 4 )$$

$${σ}_{B}^{2} = {\sum }_{c = 1}^{C} {N}_{c} ( {μ}_{c} - μ ) {( {μ}_{c} - μ )}^{T} \qquad ( 5 )$$

Where x_{ci}∈ [symbol] ^{#feat×1}∈R#feat×1 is a datapoint belonging to class c, given C total classes each with N_{c} datapoints, class means μ_{c}, and training set mean μ.

Once the new basis vectors are calculated, each new datapoint x is rebased and classified according to its maximum a posteriori probability, assuming a Gaussian distribution for each class:

$${c}_{p r e d} = {arg max}_{c} {( x - {μ}_{c} )}^{T} {\sum }_{c}^{- 1} ( x - {μ}_{c} ) \qquad ( 6 )$$

Training data for the classifier was taken in 9 separate arm positions spanning the expected 3D workspace for the experiment. Subjects were presented with visual and verbal cues for different classes in each arm position and instructed to hold each class for 2 seconds per position.

An additional majority vote post-processing filter was added at the output of the classifier.

cₒᵤₜ=mode(c_{pred}(t−L:t)) (7)

The filter length L was varied on a per-session basis during classifier training. Higher values of L improved output stability but reduced responsiveness. Experimental values of L ranged from 10-35 windows (0.5 s-1.75 s) and were set as the lowest possible value that still achieved noise-free class transitions.

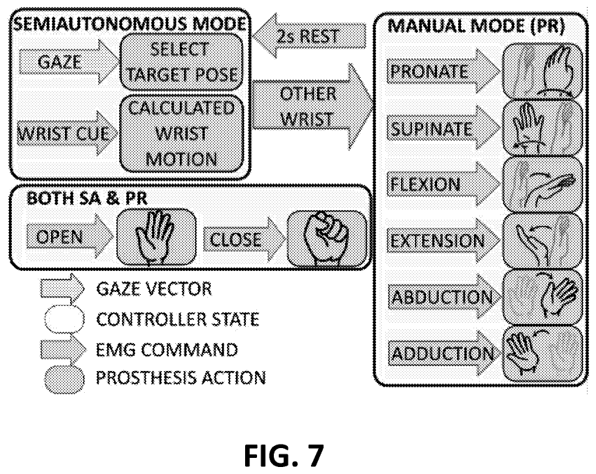

When using the PR interface, motion of the prosthetic joints corresponded directly to the output of the EMG classifier cₒᵤₜ (See Eq. 8). For the 1DOF system, subjects trained a classifier to differentiate between 5 different classes: ‘Rest’, ‘Hand Close’, ‘Hand Open’, ‘Pronate’, and ‘Supinate’. A ‘Rest’ class would maintain the current prosthesis configuration. A ‘Hand Close’ or ‘Hand Open’ command would direct the vMPL arbiter to move the hand into the specified grasp. ‘Pronate’ and ‘Supinate’ classes increase or decrease θ₁ at a fixed rate according to Eq. 8. For the 2DOF system, subjects added two additional EMG classes to control θ₂, ‘Flexion’ and ‘Extension’. At 3DOF ‘Abduction’ and ‘Adduction’ were added to control θ₃. See FIG. 7 for images of the corresponding wrist classes and motions.

θᵢ(i+1)=θᵢ(t)+sign(cₒᵤₜ)K_{q} (8)

where i is the wrist joint indicated by cₒᵤₜ, and K_{q} is an angular velocity set by the subject according to preference during training. The function sign(cₒᵤₜ) designates the direction of motion a particular class will exert on a joint (e.g. ‘Pronate’ decreases θ₁, while ‘Supinate’ increases it)

With the Semi-Autonomous interface, subjects utilize a combination of gaze direction and EMG classifier output cₒᵤₜ to command the joint angles of the vMPL. Information about the pose of the selected object is used to calculate optimal palm orientation. Because this experiment takes place in a virtual environment, all information about the environment is easily accessible to the controller. These idealized conditions allow analysis of the impact of the user interface independent of pose estimation system accuracy.

When designing the interface, it was ensured that the prosthetic joints will never move without direct input from the user. If the user disagrees with the calculated wrist motion, they are able to assume manual control of the prosthesis. Additionally, if no target pose has been selected, the interface will remain in manual mode. When in manual mode, the device responds identically to the PR interface, where the motion of the prosthesis is a direct reflection of the cₒᵤₜ class predicted by the EMG classifier. If there is no direct user activity in manual mode for more than 2 s, the interface will switch back to SA Mode, as illustrated in FIG. 7.

When no object is making contact with the palm, gaze can be used to select a target object from the environment, and the palm pose

$${g}_{s e}^{*}$$

is set to the object pose. When there is contact to the palm, placement locations can be selected from a predefined subset, and

$${g}_{s e}^{*}$$

is set to the pose associated with the selected location. A red box is drawn on the VR display to indicate the selected object or placement location. A visual cursor on the VR display indicates the estimated gaze vector and the calculated controller motion.

Once a target pose has been selected, the default mode for the interface is SA Mode. Target poses g* are indicated with the gaze vector. During training, the user selects one of the available wrist classes (e.g. “Pronate”) to designate as a wrist cue, c_{cue}. The original function of c_{cue} is replaced, and it is instead used to drive the prosthesis along a controller calculated path towards the target pose. Use of any of the other EMG wrist classes will switch the interface to Manual Mode, and then the corresponding action will be performed.

FIG. 7 illustrates a flow diagram of a workflow of the user interface with the SA controller. A ‘Rest’ class will maintain the current prosthetic configuration. A ‘Hand Open’ command will always open the hand, and a ‘Hand Close’ command will always close the hand. Subjects select one wrist command to cue calculated wrist rotation that will align the prosthesis palm with the target pose. Other wrist commands will put users into a ‘manual mode’ where prosthesis motion corresponds directly to EMG commands (as in the PR interface). A sustained ‘Rest’ command will return the user to ‘shared’ mode.

When users generate the c_{cue} EMG class in Semi-Autonomous Mode, a Resolved Rate Controller (RRC) algorithm is used to drive the available prosthetic wrist joints towards

$${g}_{s e}^{*} .$$

The velocity of the wrist motion is proportional to the pose error expressed in twist coordinates ξ:

$${q}_{t + 1} = {q}_{t} - {K}_{q} {Δ}_{t} {J}_{b}^{- 1} A ξ \qquad ( 9 )$$

where K_{q} is a constant determined by the subject, Δₜ is the period of the control loop, A is a weighting matrix selected to emphasize the rotational component of the pose error, and J_{b} is the body Jacobian relating the velocities of the controllable DOF to the end-effector twist. The i^{th} column of J_{b} can be calculated as:

where Sᵢ is as defined in Eq. 3, and Ad_{g} denotes the adjoint representation of a rigid body transformation matrix g=(R, p)∈SE(3). This is used to change the reference of a twist motion or wrench force, between two frames related by g.

$$A {d}_{9} = \vert \begin{matrix}R & 0 \\ [ p ] R & R\end{matrix} \vert ∈ {ℝ}^{6 \times 6} \qquad ( 11 )$$

$$[ p ] = \vert \begin{matrix}0 & - {p}_{3} & {p}_{2} \\ {p}_{3} & 0 & - {p}_{1} \\ - {p}_{2} & {p}_{1} & 0\end{matrix} \vert \qquad ( 12 )$$

Participating subjects were asked to perform a pick-and-place task in a VR environment, shown in FIG. 1, using either aSA or a PR controller. A total of 4 non-amputee subjects were recruited (1 female, 3 male), aged 23-29. Subjects had no known neurological disorders and varying levels of experience with myoelectric pattern recognition algorithms. All subjects had prior experience with VR platforms, and two had a known proclivity to VR-induced motion sickness. All subjects were right-handed. Informed consent was obtained from all subjects, and the study was conducted with the approval of the Johns Hopkins Medical Institutional Review Board.

During the experiment, the vMPL was superimposed on top of a subject's biological limb using a kinematic tracker. Subjects then utilized the vMPL to manipulate virtual objects in the experimental task. Subjects were presented with a total of 6 different prosthesis systems, varying both the number of available DOF in the wrist (1-3) and the interface type (PR or SA). Wrist DOF were added in an order to reflect their importance in performing Acts of Daily Living.

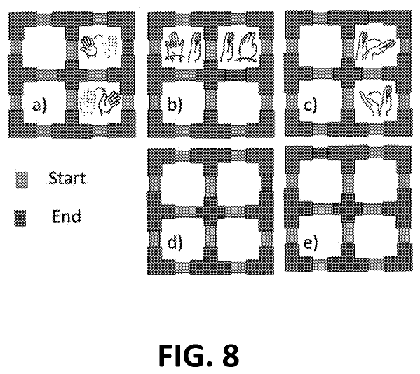

FIG. 8 illustrates a schematic diagram of the pick-and-place task. Subjects were presented with 5 types of pick-and-place tasks, each repeated 10 times in a randomized order. Three of the task types (a,b,c) were designed to encourage single-DOF activations of the wrist. Two of the tasks (d,e) were designed to elicit multi-DOF wrist activations.

To evaluate the various system designs, subjects performed a pick-and-place task on a virtual Prosthetic Hand Assessment Measure (vPHAM). The vPHAM system comprises an adjustable windowpane structure, with six horizontal and six vertical segments, each outfitted with an object holder, as illustrated in FIG. 8. The experimental task consisted of 5 different variations of pick and place task, each repeated 10 times in a randomized order. Three of the task types were designed to encourage single-DOF activations of the wrist. Two of the tasks were designed to encourage multi-DOF wrist activations. A button is also included in the frame to enable user-driven timer control. Pressing the button starts/stops a timer that is used to determine completion time for each manipulation. Physical and virtual versions of the PHAM have been used in studies of upper limb motion with non-amputee and amputee subjects, using transradial and transhumeral prostheses.

The experimental protocol followed an AB single case experimental design (SCED). Each of the recruited subjects used the SA and PR systems interchangeably for a minimum of six training sessions and six testing sessions over a period of 4-6 weeks. For Sessions Aₙ, participants operated PR systems, while for Sessions Bₙ, participants operated SA systems. Subjects trained on all 6 systems at least once in order of increasing DOF before beginning any testing sessions. Both PR and SA systems were presented to each subject the same number of times for any DOF to prevent biases from the learning effect. Half of the subjects were presented with the SA system first while the other half was presented with the PR system first, to prevent biases in user preference.

In any given session, subjects began by calibrating the EMG classifier and the gaze tracker. If the subject was able to achieve reliable EMG class access (determined if all classwise true positive predictions were ≥95%, ≥90%, ≥85%, for the 1-, 2-, and 3DOF systems respectively) and had already trained on each interface/DOF combination at least once, then the session could qualify as a testing session. On both training and testing sessions, subjects spent up to 15 minutes in a sandbox environment of randomly generated tasks. During this time they were able to make modifications to the wrist rotation speed K_{q}, c_{cue} selection, and recalibrate the classifier or gaze tracker if desired. Each task type was repeated 10×, for a total of 50 tasks presented in a randomized order. Throughout the tasks, subjects were instructed to keep their feet planted to reduce trunk motion, ensuring that most compensatory motion was restricted to the upper arm.

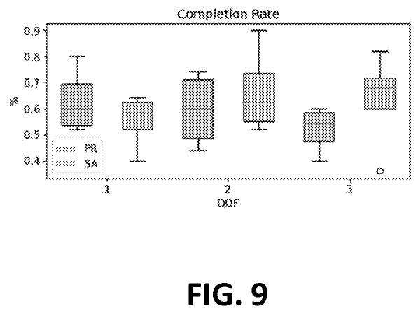

A given task was considered successful if subjects placed the object in the correct holder within a 15 s time limit without dropping it. The small sample size of the dataset (4 subjects) limits the statistical significance of cumulative metrics. Trends in the data show that with the SA system task completion rate increased with the available DOF. The PR interface showed the opposite trend, with completion rates decreasing as wrist DOF were added, as illustrated in FIG. 9. It is worth noting that the distribution of completion times for successful tasks remained relatively fixed over controller types and available DOF. This means that the increased efficiency of motion in the SA controller made up for any delays incurred by adding an extra process step of target pose selection.

FIG. 9 illustrates a graphical view of task completion rates. Task completion rates were evaluated for both interfaces types as the available prosthetic DOF was increased. Even with a limited sample size there is a trend towards increased completion rates with increased DOF when using a SA controller, but a decrease in completion rates with increased DOF with the PR controller.

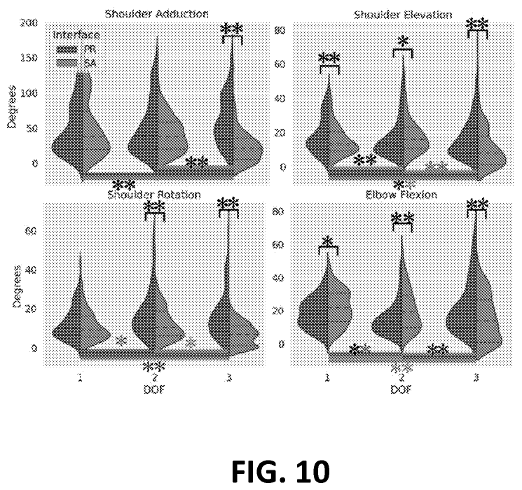

FIG. 10 illustrates graphical views of ranges of motion according to completion of tasks according to an embodiment of the present invention. For each successfully completed task the range of motion (θₘₐₓ−θₘᵢₙ) is shown for the joints in the upper arm. A lower range of motion, especially in more proximal joints, indicates increased biomechanical efficiency. * and ** denote statistical significance (p≤0.05) and (p≤0.01). A black color indicates that the significance holds for both PR and SA systems, while dark grey/light grey denote significance for that respective system.

Because the arm is a 7DOF system (even more DOF if the motion of the torso is considered) performing a 5DOF task, there is sufficient redundancy in the shoulder to overcome errors in palm orientation after wrist motion. However, due to the decreased lever arm, it is generally more biomechanically efficient for orientation changes to take place in the more distal joints (e.g. wrist), rather than at the shoulder or torso. Thus, an important measure of prosthesis performance is the amount of compensatory motion that occurs in the residual limb. In prior work the amount of compensatory motion has typically been determined by considering the range of motion for each biological joint, with a lower value signifying less compensation. The shoulder elevation angle is calculated as the angle in the sagittal plane between the vector of the upper arm and the vector of the torso. Shoulder abduction is defined as the angle of the upper arm in the transverse plane relative to the torso. Humeral rotation was calculated as rotation around the axis of the upper arm, and elbow flexion was computed as the angle between the upper arm and the lower arm.

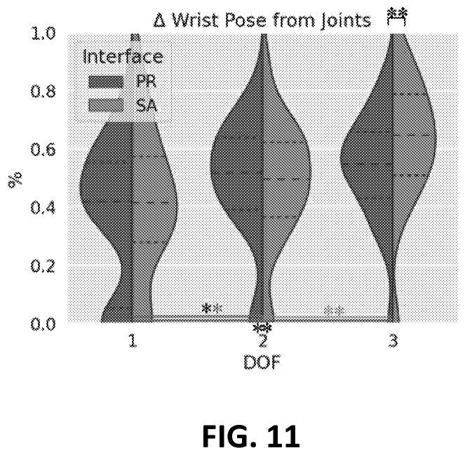

FIG. 11 illustrates graphical views of a ratio of pose changes in the wrist. For every successfully completed task, the ratio of pose changes in the wrist due to prosthetic joint motion was calculated and graphed against the total number of pose changes.

Further results show that with the SA controller there was a decrease in compensatory motion in the shoulder the DOF of the system (p≤0.01) was increased, as illustrated in FIG. 10. Statistical significance was calculated with a Wilcoxon rank-sum statistic. There was a similar decrease in compensatory shoulder adduction for the PR system from between 1 and 2 DOF. But when a third PR DOF was added, compensatory motion in shoulder elevation and rotation (p≤0.001) was worse than with less articulated systems. There was significantly less shoulder motion (p≤0.05) with the 3DOF SA system than the 3DOF PR system. At 2 and 3DOF there was an increase in the range of motion in the elbow of the SA systems as compared to the PR systems, but this is positive finding, as it is more biomechanically efficient for the motion to take place in the elbow rather than the shoulder.

Additionally, the percentage of wrist pose changes were examined to determine whether they were due to motion in the wrist joints, as opposed to motion in more proximal parts of the kinematic Chain, as illustrated in FIG. 11. The count for wrist pose changes considered instances where the difference in global rotation quaternion between frames had a norm above a noise threshold of ε=0.005. When increasing the available DOF from 1 to 2, there was a statistically significant increase in the amount of rotation due to the wrist joints for both interfaces (p≤0.05). For the SA interface, there was a further increase from 2 to 3DOF (p≤10⁻⁶), but no significant change for the PR interface.

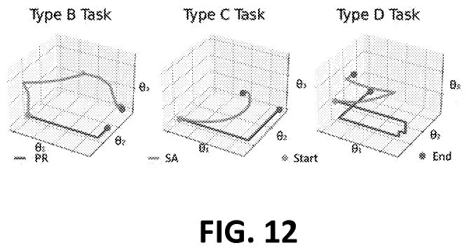

Illustrative sample trajectories for some of the different task types are show in FIG. 12. Simply by offering simultaneous joint motion, the SA controller results in more efficient motion through joint angle space, and less overall wrist joint motion (p≤0.01) for all DOF cases. Further, the percentage of inexpedient wrist motion (that actually increased pose error) was significantly lower with the SA controller as compared to the PR controller at 2 and 3DOF (p≤0.05).

FIG. 12 illustrates graphical views of sample paths through joint angle space for different task types with both the 3DOF SA and PR controller. Start locations are marked with light grey, end locations in dark grey. Because the PR interface necessitates sequential motion, it is inherently less efficient than the SA controller.

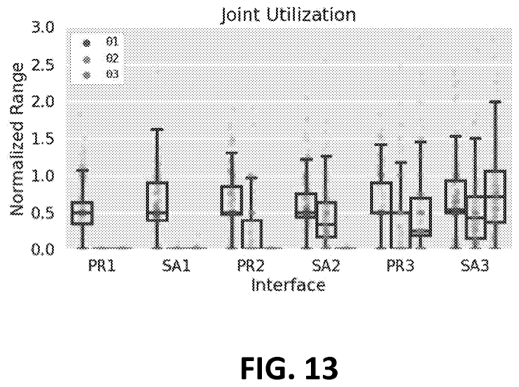

FIG. 13 illustrates a graphical view of joint utilization, according to an embodiment of the present invention. For all successful trials, the path length for each joint angle is normalized to the overall range of motion for that joint. Normalized path length for each trial are plotted as points with low opacity, and a boxplot summarizes the distribution. Even when provided more DOF, users with the PR interface will largely rely on a single joint.

To assess the accessibility of the configuration space, the utilization of the available joints for each controller configuration are considered. For all successful trials, path lengths were measured for each joint independently, and then normalized by the range of motion for the joint, as illustrated in FIG. 13. Using the SA controller, there is notably more utilization of the extra configuration space when subjects are given 2 and 3DOF (p≤10⁻⁴). With the PR interface, the median joint path length for θ₂ is 0 degrees for all DOF configurations. Further, for the PR interfaces, path lengths cluster at multiples of the joint angle range. This indicates that users would simply activate a joint until hitting the range of motion limit. In the SA controller a more even spread of values is seen, implying better graded proportional control.

The present invention increases DOF for patients using robotic upper limb prostheses and shows the importance of using semi-autonomous interfaces to make those DOF accessible. The experiment took place in a virtual environment due to the lack of availability of multi-DOF prosthetic wrists. A hybrid gaze-EMG user interface for a controller, according to the present invention, allows users to plan in task-space instead of joint-angle space. The results show a substantial (p≤0.001) reduction in compensatory motion with each added DOF when using the SA controller, while PR users stop benefiting after 2DOF.

Combined with the analysis of configuration space paths, PR users tend to rely on a single prosthetic joint, preferring biomechanically inefficient and damaging compensatory motion in the trunk and upper arm. Trends of use of the present invention show performance improvement as DOF is increased with a SA controller and decreased or saturated performance with the PR controller. This study considered the transradial case, but when extrapolating to higher levels of amputation (transhumeral, shoulder disarticulation), the value of a semiautonomous system only becomes more significant.

A notable limitation of this study is that the subject pool consists entirely of people with intact limbs. Individuals with intact limbs typically exhibit superior performance with EMG-classification systems compared to persons with some degree of upper limb loss. Thus, the performance gap between the PR and SA controllers would only grow with a more targeted subject population. Now that the value of fully articulated wrists and semi-autonomous interfaces has been validated, future work in this area includes transitioning to a physical testing platform and recruitment of a more clinically relevant subject pool. Additionally, examination of bio-mimetic or other trajectory planning methods is an open problem, especially for systems that include an elbow or shoulder joint, which play a more significant role in palm placement as well as orientation.

Shared control has emerged as a promising alternative system paradigm. In this approach, control is distributed between the user and an autonomous algorithm that predicts the user's motion intent. Thus, the autonomous controller can manage low-level motion planning and allow the user to concentrate on high-level decisions, such as reaching and grasping. The majority of this work has examined use of computer vision (CV) systems to classify objects and determine optimal grasp type. A handful of studies have included some object-pose estimation that can be used to orient a 1-2 DOF wrist. As discussed above in some embodiments this work can be applied to a semi-autonomous transradial prosthesis interface that demonstrates effective use of a 4 DOF system. However, to date, there has not been any investigation of shared control systems for patients with higher levels of amputation, who stand to benefit more from the framework.

In parallel, Dynamic Movement Primitives (DMPs) have shown promising results for generating smooth and adaptable trajectories from demonstrations while maintaining stability guarantees. Essentially, DMPs combine a proportional derivative (PD) controller that ensures stability with a nonlinear forcing term that captures demonstration variation. A canonical system is employed for temporal synchronization and autonomous execution. In many implementations of Movement Primitives, DMPs ensure precise initial robot position and velocity, thereby allowing smooth transitions between sequential goals.

There are numerous variants of DMPs, some of which have already been applied to lower-limb prostheses and rehabilitation exoskeletons. These DMPs can generally be categorized into deterministic and probabilistic methods. Deterministic approaches often modify the classical DMP formulation: adjusting the transformation system, canonical system, or radial basis functions—to enhance generalization, accuracy, and computational efficiency. In contrast, probabilistic methods (e.g., Gaussian Mixture Models) represent trajectory distributions, enabling them to handle variability in human motion and generalize to new tasks similar to the initial demonstrations. However, the inherent uncertainty in probabilistic execution can obscure the interpretability of characteristics in the generated trajectories.

The deterministic DMP can also handle the variations in multiple demonstrations. The extended DMP proposed in employs a roto-dilatation matrix and aligns all demonstrations via linear regression to extract a common behavior from demonstrations. A second approach is to learn many sets of weights, and use different task parameters p to select the relevant subset. The correct weights for a new task can be selected discretely so that w_{1:N}=Φ(p), with a common set of activations ψ, or projected onto a continuous space, allowing for multiple sets of basis functions in the same formulation.

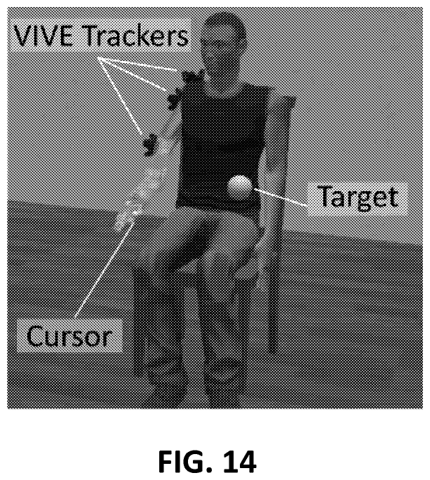

The present invention uses a linear regression model for learning DMP basis weights to generate bio-mimetic upper limb trajectories. While not generalizable to all tasks, this method is surprisingly effective for a reach-to-point task. FIG. 14 illustrates a schematic view of a setup for the present invention. Participants utilize a virtual transhumeral prosthesis to perform a reach-to-point task. This requires collaborative motion of the participant's shoulder and a robotic elbow. Kinematic trackers are used to overlay the virtual prosthesis on the participant's arm, provide real-time estimation of the shoulder joint configuration, and evaluate performance.

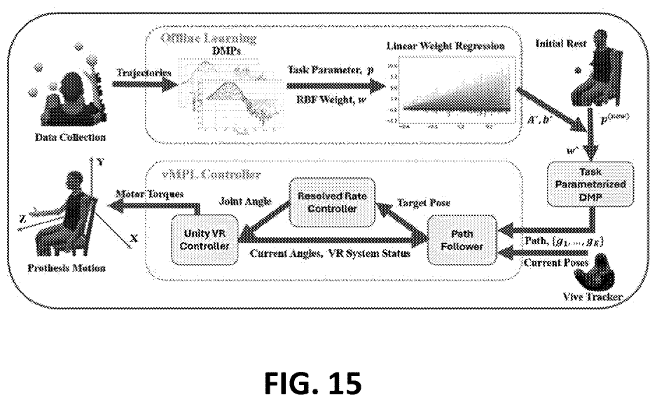

The initial analysis is performed on a dataset collected from six able-bodied participants and one amputee, and performance is compared against both a Kernel-based mixture of motor primitives and the extended DMP. Real-time human in the loop evaluations were also conducted in a virtual reality (VR) environment. FIG. 15 illustrates a flow chart of the proposed method, from data collection through DMP training and online execution. This work contributes to the literature in three major ways: 1) It is the first study examining a semiautonomous controller for transhumeral upper limb prosthesis. 2) The present invention includes a method for generating bio-mimetic trajectories that can generalize across the entire reach-space using relatively few demonstrations from a sparse sampling of the space. 3) Three representative approaches to task-parameterized DMPs are tested and the key factors that affect the fidelity of the generated trajectories are analyzed.

DMPs are a widely adopted Learning by Demonstration frameworks. For discrete motions, the transformation system is as follows:

r\dot{v}=K(g−x)−Dᵥ−K(g−x₀)s+Kf(s) (13a)

r\dot{x}=v (13b)

where x and v denote position and velocity, g is the goal, τ controls temporal scaling, and K, D are stiffness/damping coefficients in a spring-damper analogy. The term −K(g−x₀)s maintains the ability to scale the DMP based on the distance between initial and goal positions while also mitigating instabilities when g is near x₀. Compared to classical DMP formulations, this alternative structure avoids instabilities when g≈x₀ by eliminating the (g−x₀) multiplier in the forcing term ƒ(s). This situation frequently occurs in human motion when moving in a plane or a line (i.e. g≈x₀ for 1-2 DOFs).

Both the classical and this alternative formulations rely on a canonical system to govern temporal synchronization:

τ\dot{s}=−αs (14)

where s (initialized at 1) decays autonomously to 0. This removes explicit time dependence from the forcing function ƒ(s) and allows temporal scaling of motions through adjusting τ, α. As s approaches to zero, the forcing term naturally vanishes, ensuring the system converges to the goal. To capture the shape of a demonstrated trajectory, the forcing function ƒ(s) takes the form:

$$f ( s ) = {\sum }_{i}^{N} \frac{{w}_{i} {ψ}_{i} ( s ) s}{{\sum }_{i}^{N} {ψ}_{i} ( s )} \qquad ( 15 a )$$

$${ψ}_{i} ( s ) = exp ( - {{h}_{i} ( s - {c}_{i} )}^{2} ) \qquad ( 15 b )$$

where {wᵢ} are learned weights, and {ψᵢ(s)} are a set of N bases, typically Gaussian radial basis functions (RBFs), although others are possible (e.g. truncated kernels). The heights and widths of basis functions are set heuristically; classically, this results in a uniform distribution over time. Learning proceeds by computing the target forcing term from recorded {x(t), v(t)} data using Eq. (15) and Eq. (13) via locally weighted regression or similar methods.

FIG. 15 illustrates a schematic diagram of the controller and offline learning process. To create a generalized model for predicting reach-to-point trajectories, the proposed Linear Weight Regression DMP is trained using a sparse sampling of demonstrations from the task space. Individual models are created for each of these demonstrations. Then, a regression approximates the basis weights as a function of the parameter space (Cartesian coordinates for this application). This model can be used to create DMPs that generate bio-mimetic trajectories from rest to any point in the human reach-space. A trajectory follower can then be used to guide the actuation of a robotic prosthetic elbow in conjunction with the user's shoulder motion.

The classical DMP formulation often operates in a one-shot paradigm, where a single demonstration defines the nonlinear forcing term. While multiple trajectories can be combined through concatenation and learned via locally weighted regression, such direct merging tends to average out variations among the demonstrations. In particular, when the initial or goal positions differ across demonstrations, the basic model does not explicitly account for these differences.

To generalize across reach-space goals, a linear mapping between task parameters p∈ [symbol] ^{d} (i.e. Cartesian goals) and RBF weights w∈ [symbol] ^{N} is proposed. For M demonstrations with task parameters {p^{(j)}} and fitted weights {w^{(j)}}:

w=Ap+b (16)

where A∈ [symbol] ^{N×d} and b∈ [symbol] ^{N} are regression constants computed via least squares from M learned DMPs,

$${\{ {p}^{( j )} , {w}^{( j )} \}}_{j = 1}^{M} .$$

In particular, A and b are determined by minimizing the sum of squared residuals between the observed weight vectors w^{(j)} and their estimates Ap^{(j)}+b:

$${min}_{A , b} {\sum }_{j = 1}^{M} {‖ {w}^{( j )} - ( A {p}^{( j )} + b ) ‖}^{2} \qquad ( 17 )$$

The optimal parameters, A* and b*, then enable the generation of task-specific forcing terms:

$$f ( s ; p ) = {\sum }_{i}^{N} \frac{{( {A}^{*} p + {b}^{*} )}_{i} {ψ}_{i} ( s ) s}{{\sum }_{i}^{N} {ψ}_{i} ( s )} \qquad ( 18 )$$

In a standard DMP framework, a phase variable governs temporal progression, linking the motion's execution time to the learned trajectory. However, in the transhumeral prosthesis setting, human arm motion can vary significantly, and the manipulator base (the shoulder) is itself “floating” rather than fixed. Although one could estimate the motion duration (i.e., phase) from recognition algorithms, such approaches can be computationally intensive and less reliable for real-time control.

To address these synchronization challenges, the DMP is employed primarily as a high-level motion guide rather than a strict time-parameterized trajectory generator. For each new task (defined by start and goal positions), the DMP uses regression-derived weight vectors to produce a coarse Cartesian trajectory. By choosing a smaller temporal scaling factor r, the DMP outputs K discrete waypoints:

{g₁,g₂, . . . ,g_{K}}⊂ [symbol] ³.

These waypoints capture the general path in 3D space without enforcing exact timing.

Because the transhumeral prosthesis operates within a reduced workspace compared to a typical human arm, many targets in 3D space may lie beyond its reachable range. To handle such situations, a Resolved-Rate Controller (RRC) is used, which is more robust to out-of-reach targets. Let x(t)∈ [symbol] ³ be the current end-effector position and q(t) the joint configuration at time t. J(q(t))∈ [symbol] ^{3×n} is the corresponding Jacobian. The controller proceeds as follows:
1) Identify the nearest waypoint gᵢ to the current end-effector position:

$$i = arg {min}_{i ∈ [ 1 , … , K - 1 \}} ‖ x ( t ) - {g}_{i} ‖$$

2) Select the next waypoint, gᵢ₊₁, as the immediate target.
3) Compute the next joint configuration q(t+Δt) via

q(t+Δt)=q(t)+J^{†}(q(t))(gᵢ₊₁−x(t))αΔt

where J^{†}(⋅) is the (pseudo-)inverse of the Jacobian and a is a proportional gain to scale the position deviation, (gᵢ₊₁−x(t)).

Once gᵢ₊₁ becomes the closest waypoint to the current end-effector position, the index advances to i+2 and the process repeats until the final waypoint g_{K} is the next position to be selected. Although this approach does not preserve the exact timing profile of the original demonstration, the near-uniform spacing of the waypoints ensures a reasonably direct approximation of the intended trajectory.

The dataset for model training and initial offline analysis was drawn from a prior Institutional Review Board approved study at the Johns Hopkins University School of Medicine. It included six participants with intact limbs (four males and two females, aged 25-29, all right-handed) and one 35-year-old male participant who experienced partial upper limb loss (ULL) due to complications from meningitis.

Each participant performed a 3D endpoint acquisition task in a VR setting. While seated and wearing a head-mounted display, participants were instructed to reach for 100 target positions uniformly distributed within a spherical section around the dominant shoulder, with preset angular and radial boundaries. The start position for all tasks was located at the joint-angle-space origin. This is the lowest-energy limb configuration with a singularity that allows us to enforce a consistent point in joint angle space, which is useful for comparing performance across trials and participants. HTC VIVE trackers were placed on the shoulder, elbow, wrist, and hand to capture kinematics data at 90 Hz.

In this study, the shoulder is regarded as the fixed base of the dominant limb. The present invention calculates hand trajectories expressed in shoulder-centered coordinates. Any demonstration that fell outside the defined reaching region or exceeded a time threshold (approximately 2 s, with a slightly more lenient criterion for the amputee) was excluded from further analysis, which left 519 trajectories in total, 54 of which were from the ULL participant. Static motion segments (velocity <0.08 m/s) were trimmed to focus DMP training on active reaching stages. A second-order Butterworth lowpass filter at 6 Hz was applied bidirectionally to reduce high-frequency noise without introducing phase lag. Finally, all demonstrations were time-aligned to ensure uniform duration based on the observation that task completion times were generally consistent across participants.

To evaluate performance, how closely each reconstructed trajectory matches its original demonstration ix measured by computing the Dynamic Time Warping (DTW) distance. DTW finds an optimal alignment between two time series and quantifies their similarity in terms of overall trajectory shape. This method is useful for comparing motion patterns that exhibit local phase shifts or variations in velocity, because it calculates the minimal cumulative distance needed to align the two trajectories point by point.

To validate the feasibility of prosthesis control approach, the Linear Weight Regression DMP framework is integrated into a real-time, VR-based prototype. In the system of the present invention, the DMP generates coarse waypoints that guide a resolved-rate controller, which in turn governs the motion of a simulated transhumeral prosthesis. Four participants (2 males, 2 females, aged 19-36) were recruited to assess the usability and performance of the generated trajectories in the context of a semi-autonomous transhumeral prosthesis. Prior to participation, informed consent was obtained from all subjects, and the study protocol was approved by the Johns Hopkins School of Medicine Institutional Review Board.

Participants performed a 3D endpoint acquisition task similar. All 100 goal points in the online experiment were independent from the training set, generated pseudo-randomly to span the reach space. Instead of a conventional hand cursor, the task displayed a cursor located on the palm of a virtual transhumeral prosthesis, as illustrated in FIG. 14. Because all participants had intact upper limbs with no known motor deficits, natural limb motions are also recorded as a baseline for comparison. Pose information was collected via kinematic trackers attached to the shoulder, upper and lower humerus, forearm, wrist, and hand. The virtual prosthesis was overlaid on each participant's arm by referencing the lower humerus tracker.

To evaluate system performance, three trajectories were compared: (1) the participant's natural limb motion, (2) the DMP-generated trajectory (i.e., the coarse Cartesian waypoints), and (3) the actual path executed by the resolved-rate controller in the VR environment. Specifically, DTW was used to compute the minimal cumulative distances among these trajectories.

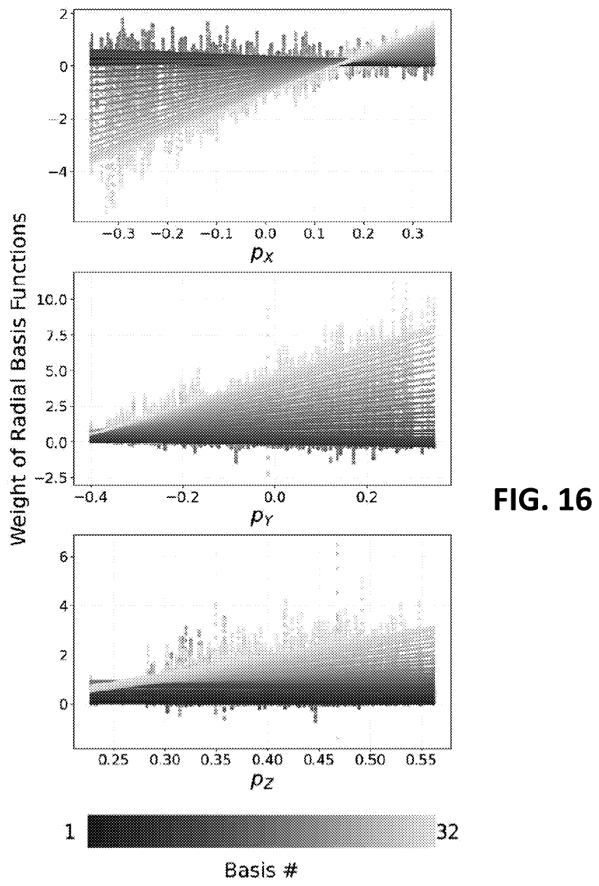

The crux of the proposed Linear Weight Regression DMP framework lies in the observation of a linear relationship between the weights of the DMP forcing terms and the Cartesian coordinates of reach-to-point tasks, as illustrated in FIG. 16. To validate this hypothesis, DMP models were trained (32 bases, K=150, D=2√K, α=2) on 519 trajectories across each dimension of motion in Cartesian space. A linear regression model was then applied to fit the distribution of weight vectors over task parameters. The results revealed a median coefficient of determination R² of 0.63 (horizontal X-axis), 0.43 (vertical Y-axis), and 0.02 (depth Z-axis). 97% (X), 94% (Y), and 84% (Z) of the regressions were statistically significant (p<0.05), compared to a null hypothesis of zero slope. These findings suggest that linear models effectively approximate DMP weights for horizontal and vertical motion while struggling to capture depth-related variations.

FIG. 16 illustrates graphical views of linearity of RBF weights. Each point represents one of 32 weights learned for each demonstration, each corresponding to a basis in the forcing function. When plotted against the Cartesian goal coordinates, a strong linear trend is noted across reach tasks, displayed as lines of best fit for each basis. This can be used to predict weight values for unobserved motions.

The diminished performance in the depth axis likely stems from inherent complexities in human motion. Unlike horizontal and vertical movements, depth adjustments (Z-axis) often involve non-linear kinematic constraints, such as detours around the torso or compensatory shoulder motions

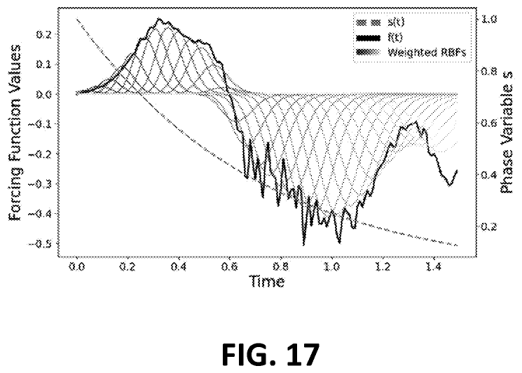

FIG. 17 illustrates a graphical view of an exemplary distribution of unnormalized RBFs (i.e. φᵢ(s)s(cᵢ)). These values reflect a DMP model for one particular demonstration. A weighted sum of evenly-distributed RBFs forms a nonlinear forcing function to model a given trajectory. The color indices of bases are the same as FIG. 16. The phase variable s, which parametrizes the forcing function, decays autonomously over time. The RBFs cancel each other to achieve the zero value of forcing term at mid stage.

Additionally, perceptual challenges in virtual reality—where depth estimation is less precise—may introduce overshooting or corrective motions that defy linear modeling. In contrast, the strong R² values for X and Y axes indicate that linear regression sufficiently captures the dominant acceleration profiles in the horizontal and vertical movement. For instance, the upward trend in late-stage Y-axis weights, as illustrated in FIG. 16, reflects increased acceleration demands for higher targets. The fixed-hyperparameter PD controller requires the nonlinear forcing term to achieve higher speeds as targets ascend.

Further analysis revealed a stage-dependent trend in the linear model performance. While early and late movement stages (characterized by acceleration and deceleration) showed robust linear correlations (R²>0.5), mid-stage weights exhibited near zero explanatory power (R²≈0). This phenomenon aligns with the DMP's dynamics: during mid-stage, where forcing term is near zero, as illustrated in FIG. 17. In this region the PD controller dynamics dominate the overall acceleration and motion stabilization, while the forcing function is largely capturing measurement noise and other confounding factors. Consequently, mid-phase estimation of weight values is both more abstruse and less informative. Because the forcing term does not contribute to the motion significantly, any impact on trajectory accuracy is mitigated by the controller's inherent stability.

Four different approaches were analyzed with K=150, D=2√K, α=2, and 32 RBFs for DMP generalization: three multi-demonstration methods—Linear Weight Regression DMP (the proposed approach), Extended DMP, and a Kernel-based Mixture of Motor Primitives (with a standard deviation of 0.1)—as well as a single-demonstration DMP baseline learned using a central goal position from the training dataset. The two multi-demonstration methods were selected for their distinct strategies in handling multiple demonstrations while retaining deterministic strength, thus providing basic benchmarks against which the method can be compared.

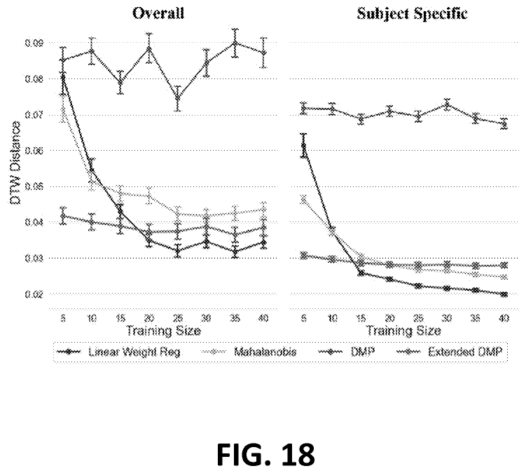

FIG. 18 illustrates graphical views of Overall Generalization Performance (left). Using 5-fold cross-validation on the combined dataset, mean DTW distance-deviations were calculated from actual trajectories (with standard errors). Each fold included an increasing number of training demonstrations to illustrate how additional data impacts performance. With sufficient demonstrations, the Linear Weight Regression DMP achieved the lowest errors. Subject-Specific Training (right). Per-subject DTW errors show that training exclusively on each individual's data improves generalization and reduces variability.

Using 5-fold cross-validation on 100 different goal positions (forming a pooled dataset of 519 trajectories; 465 from intact-limb participants and 54 from the participant with ULL), the DTW distance between the generated trajectories and the baseline demonstrations were calculated. As shown in FIG. 18 (left), Linear Weight Regression DMP outperforms both the Extended DMP and the Kernel-based approach when provided with more than approximately 20 demonstrations (with p<0.01 and Common Language Effect Sizes=0.47, 0.62, and 0.92) compared to Extended DMP, the Kernel-based method, and the single-demonstration DMP baseline, respectively. The single-demonstration baseline exhibited the highest DTW error, emphasizing the need for multi-demonstration models.

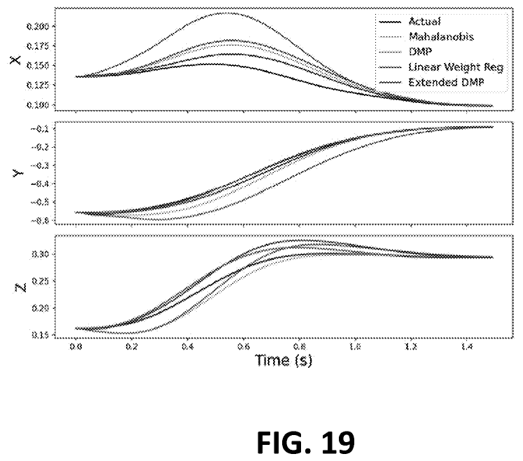

FIG. 18 (right) presents subject-specific results. Training exclusively on data from an individual participant significantly reduces both the DTW error and its variance (except for the single-demonstration method), suggesting that inter-subject differences may degrade performance when using a global model. Consequently, subject-specific or personalized calibration appears to be a worthwhile avenue for improving motion accuracy. FIG. 19 illustrates a representative trajectory from the amputee participant, comparing the method to the other DMP generalization approaches. In the example trajectory shown in FIG. 19, the Linear Weight Regression DMP achieves better alignment than the other DMP generalization approaches, yielding a path closer to the natural motion.

The better performance of the Linear Weight Regression DMP likely stems from its global mapping strategy. In contrast, the Kemel-based approach depends on local distance metrics in task space, making it sensitive to outliers or noise in few demonstrations in the corresponding regions. Meanwhile, the Extended DMP compresses all demonstrations into a single parameter set and generalize using the roto-dilatation matrix, which can be advantageous when data are limited, but may sacrifice individualized variability in trajectory shape.

FIG. 19 illustrates graphical views of a trajectory reproduction example. This representative example from an amputee participant compares the actual hand path (black) to multiple DMP-based generalization methods. Notably, the Linear Weight Regression DMP (purple) shows better alignment in the X and Y dimensions, resulting in a closer overall match to the natural hand path.

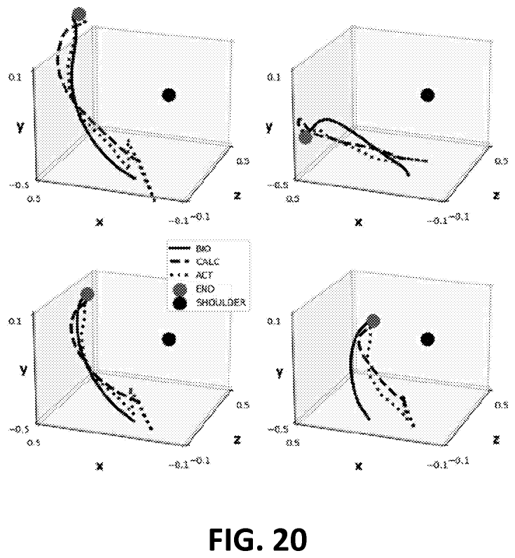

FIG. 20 illustrates graphical views of sample trajectories from the online experiment. These sample trajectories demonstrate that Linear Weight Regression DMP models, combined with a trajectory follower, allow a transhumeral prosthesis to emulate natural limb motion (mean 8.5% error) for unseen points spanning the reach space.

The median minimum cumulative distance between the calculated trajectories and the natural limb trajectories was 0.097 meters, with a variance of 0.0319 meters. With a human in the loop the realized hand trajectories were significantly more similar to natural limb motion (p<0.001), with a median minimum cumulative distance of 0.0733 meters, and a variance of 0.0284 meters. Compared to the overall length of each path, this represents a mean 8.5% error±5%. Representative samples are shown in FIG. 20.

Looking forward, the research will focus on two key directions. First, task complexity will be increased. The current study used a fixed initial position for all tasks in an empty space. While this is valuable for motion characterization, everyday activities often pose more challenges such as more diverse initial position and obstacle avoidance. By incorporating additional task parameters—like orientation and movement speed—the approach can be generalized to a wider range of realistic, complex tasks.

Secondly, testing can be done on higher DOF prostheses and can be transitioned from virtual prototypes to physical platforms. Real-world validation of sophisticated devices will allow us to address practical challenges such as mechanical constraints and sensor noise. These experiments are crucial for demonstrating the robustness and applicability of the strategy in everyday scenarios, ultimately contributing to the development of more effective and user-friendly prosthetic systems.

Prosthesis usability remains a critical challenge, especially for individuals with upper limb loss proximal to the elbow. In this work, a bio-inspired shared control framework leverages linear regression to generalize human-like reaching motions across a 3D workspace. By training DMPs on 20 demonstrations, the method achieved lower trajectory errors than some existing multi-dimension DMP-based methods, while preserving general motion patterns. Real-time evaluations further demonstrated reliable performance for the cooperative task that is reach-to-point motion using a user's shoulder and a robotic elbow. This study represents the first shared-control system tailored for transhumeral prostheses, providing a novel approach in prosthetic autonomy for high-level amputation. Future work will explore diverse tasks and validate physical prototypes, further expanding the findings to advance prosthetics and broader human-robot collaboration in a data-efficient, computational inexpensive, and user-centered approach.

It should be noted that aspects of the present invention, its control, and calculations can be executed with a program(s) fixed on one or more non-transitory computer readable medium. The non-transitory computer readable medium can be loaded onto a computing device, microprocessor, servo, server, actuator, device processor, smartphone, tablet, phablet, or any other suitable device known to or conceivable by one of skill in the art.

It should also be noted that herein the steps of the method described can be carried out using a computer, non-transitory computer readable medium, or alternately a computing device, microprocessor, or other computer type device independent of or incorporated with an imaging or signal collection device. The computing device for executing the present invention can be a completely unique computer designed especially for the implementation of this method. Indeed, any suitable method of analysis known to or conceivable by one of skill in the art could be used. It should also be noted that while specific equations are detailed herein, variations on these equations can also be derived, and this application includes any such equation known to or conceivable by one of skill in the art.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape.

It should be noted that the software associated with the present invention is programmed onto a non-transitory computer readable medium that can be read and executed by any of the computing devices mentioned in this application. The non-transitory computer readable medium can take any suitable form known to one of skill in the art. The non-transitory computer readable medium is understood to be any article of manufacture readable by a computer. Such non-transitory computer readable media includes, but is not limited to, magnetic media, such as floppy disk, flexible disk, hard disk, reel-to-reel tape, cartridge tape, cassette tapes or cards, optical media such as CD-ROM, DVD, Blu-ray, writable compact discs, magneto-optical media in disc, tape, or card form, and paper media such as punch cards or paper tape. Alternately, the program for executing the method and algorithms of the present invention can reside on a remote server or other networked device. Any databases associated with the present invention can be housed on a central computing device, server(s), in cloud storage, or any other suitable means known to or conceivable by one of skill in the art. All of the information associated with the application is transmitted either wired or wirelessly over a network, via the internet, cellular telephone network, RFID, or any other suitable data transmission means known to or conceivable by one of skill in the art.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A system comprising:
an electromyography (EMG) sensor;
a gaze tracker;
a pose tracker;
a processing device, wherein the processing device is programmed to receive data from the EMG sensor, the gaze tracker, and the pose tracker, such that the processing device generates a trajectory for a prosthetic device from the data from the EMG sensor, the gaze tracker, and the pose tracker.

2. The system of claim 1 wherein dynamic motion primitives (DMPs) are used to generate the trajectory for the prosthetic device.

3. The system of claim 1 wherein the prosthetic device comprises a transhumeral prosthesis.

4. The system of claim 1 wherein the prosthetic device comprises an upper limb prosthesis.

5. The system of claim 1 wherein the trajectory is determined in a task-based space.

6. The system of claim 1 further comprising a prosthetic device.

7. The system of claim 1, wherein the system is configured to communicate with an existing prosthetic device.

8. The system of claim 1, wherein the gaze tracker is configured to recognize known objects.

9. The system of claim 1, wherein the pose tracker is configured to detect in 6D.

10. The system of claim 1 wherein a detection of a gaze fixation point is within a predetermined segment for the target object.

11. The system of claim 1 further comprising a training mode.

12. A prosthetic system comprising:
a prosthetic device;
an electromyography (EMG) sensor;
a gaze tracker;
a pose tracker;
a processing device, wherein the processing device is programmed to receive data from the EMG sensor, the gaze tracker, and the pose tracker, such that the processing device generates a trajectory for the prosthetic device from the data from the EMG sensor, the gaze tracker, and the pose tracker.

13. The system of claim 1 wherein dynamic motion primitives (DMPs) are used to generate the trajectory for the prosthetic device.

14. The system of claim 1 wherein the prosthetic device comprises a transhumeral prosthesis.

15. The system of claim 1 wherein the prosthetic device comprises an upper limb prosthesis.

16. The system of claim 1 wherein the trajectory is determined in a task-based space.

17. The system of claim 1, wherein the system is configured to communicate with an existing prosthetic device.

18. The system of claim 1, wherein the gaze tracker is configured to recognize known objects.

19. The system of claim 1, wherein the pose tracker is configured to detect in 6D.

20. The system of claim 1 wherein a detection of a gaze fixation point is within a predetermined segment for the target object.
